(19) United States
(12) Patent Application Publication
(10) Pub. No.: US 2008/0269618 A1
(43) Pub. Date: Oct. 30, 2008

(54) Non-Invasive In-Situ Detection Of Malignant Skin Tissue And Other Abnormalities Using Laser Plasma Spectroscopy

(76) Inventors: Michael J. Myers, Hilton Head, SC (US); John D. Myers, Hilton Head, SC (US); Jeffrey A. Myers, Hilton Head, SC (US); Abbey G. Myers, Hilton Head, SC (US)

Correspondence Address: Randall L. Shoemaker;Dickinson Wright, PLLC, 38528 Woodward Ave, Ste. 2000, Bloomfield Hills, MI, 48304-2970, US

(21) Appl. No.: 12/110,528

(22) Filed: Apr. 28, 2008

Related U.S. Application Data
(60) Provisional application No. 60/914,083, filed on Apr. 26, 2007.

(51) Int. Cl. A61B 6/00; A61B 10/02

(52) U.S. Cl. 600/477; 600/562

(57) ABSTRACT

Disclosed is a system and method for a non-invasive method for determining the presence or absence of cancerous cells in the skin and deeper tissue levels. The system includes a portable handheld laser coupled with a spectroscopy system to produce real-time material analysis of the presence of cancerous cells without sample preparation. The system focuses a high peak power laser pulse onto a targeted material to produce a laser spark or micro-plasma. Elemental line spectra emission is created, collected and analyzed by a spectrophotometer. The line spectra emission data is quickly displayed on a laptop computer. “Eye-safe” Class I lasers provide for practical in-situ laser plasma spectroscopy applications such as detection of cancerous skin tissues. The emission data can be used to detect changes in the levels of a series of elements that are associated with cancerous cells versus normal skin cells. The system also finds use during excisional biopsy procedures to ensure that all cancerous cells have been removed.

### RELATED APPLICATIONS

This application claims the benefit of U.S. Provisional Application No. 60/914,083 filed Apr. 26, 2007.

### STATEMENT REGARDING FEDERALLY SPONSORED RESEARCH

NONE

### TECHNICAL FIELD

This invention relates generally to non-invasive methods for detecting changes in skin cells associated with development of skin cancer and, more particularly, to a laser plasma spectroscopy system that can be used to detect changes in the levels of several elements found in skin cells where these changes are associated with the development of skin cancers.

### BACKGROUND OF THE INVENTION

The usual screening procedure for detecting skin cancer is a visual examination of the skin and an invasive tissue biopsy. A doctor examines suspected skin areas noting the size, shape, color, texture, and if there is bleeding or scaling of any abnormalities. The doctor performs a biopsy of the suspected skin area to determine if cancer is present and if it is benign or malignant cancer. This involves the cutting and removal of skin tissue for examination under the microscope to determine if cancerous cells are present. If a skin tissue sample is found to contain cancer, the doctor will typically remove the entire cancerous area in what is called an excisional biopsy technique. During an excisional biopsy, the doctor will continue to remove tissue samples and carefully examine the margins of each sample under a microscope. Tissue samples are removed from the excision region until the tissue margins are determined to be free of cancerous cells. This involves laborious sample preparation and evaluation procedures associated with tissue examination and margin reading under a microscope. Sometimes it can be difficult to determine when the entire cancerous region has been removed and may result in excess tissue removal to ensure complete removal. In addition, this examination takes time and prolongs the duration of the removal operation.

It is very desirable to develop a non-invasive procedure for the determination of whether a suspected area of skin contains cancerous cells. In addition, it is desirable to shorten the time required to evaluate a suspected region to determine the presence or absence of cancerous cells. Finally, a real time analysis process would be useful to shorten surgical excisional procedures and to ensure that all of the cancerous cells have been removed.

### SUMMARY OF THE INVENTION

In general terms, this invention provides a non-invasive in-situ rapid method for screening skin and tissue to detect the presence of cancerous cells. More specifically the invention provides for the use of a pulsed high peak power eye-safe laser to create a plasma spark “air breakdown” at the surface of the skin and collection and analysis of the elemental line spectra thereby produced to determine the level of at least one marker element. Changes in the levels of specific marker elements are associated with the development of cancer in cells. Other elements do not show changes in cellular levels in cancerous cells, thus these elements can serve as internal control elements. The system permits for rapid detection of cancerous cells and can also be used during excisional procedures to ensure that all of the cancerous cells have been removed from a site.

With the use of an eye-safe in-situ laser plasma spectroscopy device the skin cancer biopsy may be replaced by a non-invasive in-situ screening of areas of suspected skin cancer. With in-situ laser plasma spectroscopy excisional biopsy margins may be evaluated for cancerous cells with instantaneous results.

These and other features and advantages of this invention will become more apparent to those skilled in the art from the detailed description of a preferred embodiment. The drawings that accompany the detailed description are described below.

### DETAILED DESCRIPTION OF THE DRAWINGS

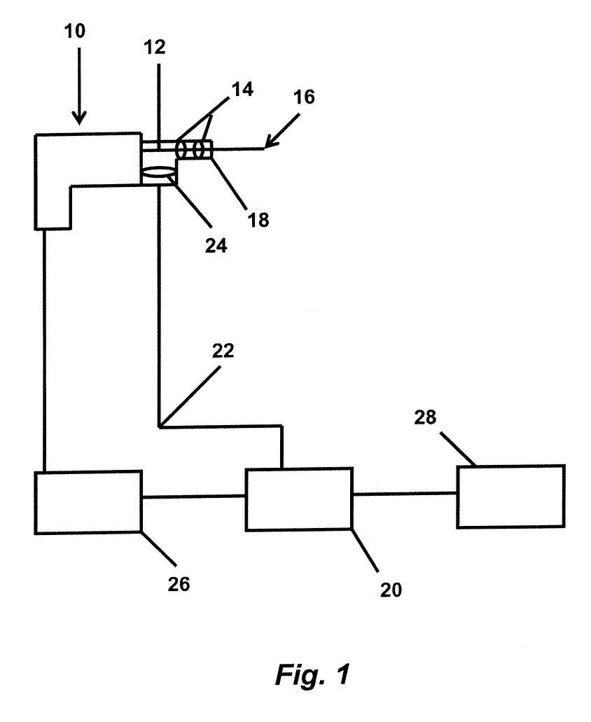

FIG. 1 is a schematic diagram of a detection system according to the present invention;

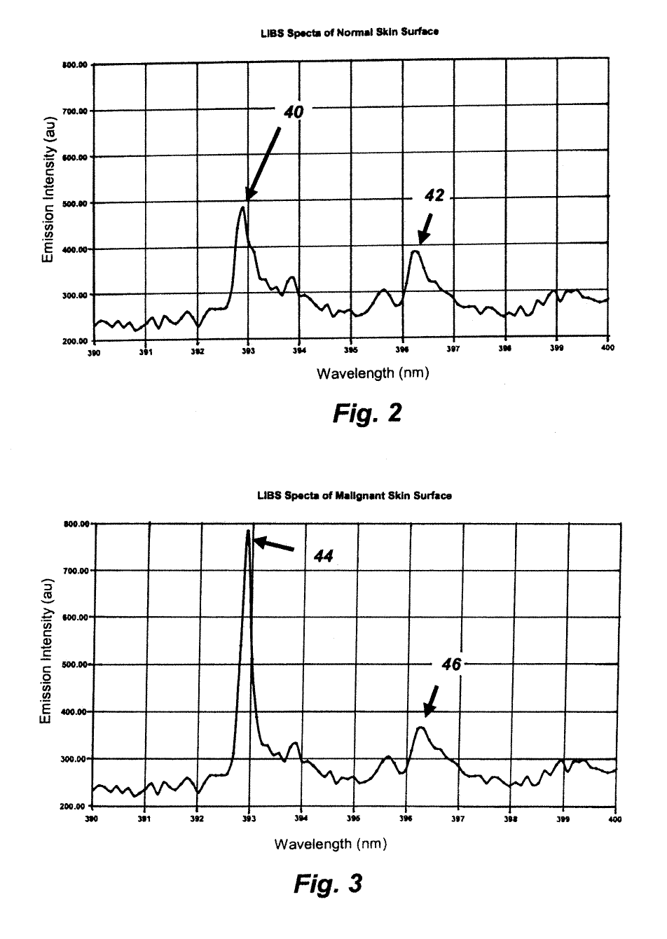

FIG. 2 is a trace showing a line spectra emission of the elements calcium and aluminum from normal healthy skin cells; and

FIG. 3 is a trace showing a line spectra emission of the elements calcium and aluminum from cancerous skin cells.

### DETAILED DESCRIPTION OF A PREFERRED EMBODIMENT

The present invention is directed toward a method and device for non-invasive in-situ comparative quantitative determination and detection of cancerous skin tissue cells using laser plasma spectroscopy. When healthy skin tissue cells become cancerous, there are changes in the cellular levels of certain marker elements such as calcium, zinc and iron in the outer skin cell layers. By measuring the changes in levels of certain marker elements associated with the growth of cancerous skin tissue, one can identify the cancerous areas of skin. For example, on the human skin surface, down to the first 50 microns of depth, the cellular level of calcium increases dramatically in cancerous cells by a factor of about 2× while the cellular level of aluminum remains constant even in cancerous cells. Thus, the element calcium can serve as a marker element for cancerous cells while aluminum is an internal control element. Other cancer marker elements include iron and zinc which also exhibit substantially increased levels in the outer layers of cancerous skin cells when compared to the outer layers of healthy skin cells. The cellular level of iron increases by a factor of about 2× while the cellular level of zinc increases by a factor of about 3× in cancerous skin cells that are at the surface or within 50 microns of the surface. The levels of these cancer marker elements may be compared to the levels of stable internal control elements such as aluminum, potassium, magnesium or other internal control elements present in the outer skin cell layers that exhibit no significant change in cellular level when the cell becomes cancerous. The method of the present invention is very sensitive and allows for the determination of elements even if they are present at only parts per million (ppm) levels.

The system of the present invention is shown in a schematic diagram in FIG. 1. The present invention includes a small hand held high peak power plasma spark source such as a Q-switched pulsed laser with greater than 0.1 megawatt per pulse output power, shown at 10. The laser output pulse 12 is sent through conditioning optics 14 that focus the pulse to a small laser focus point 16 a set distance from the laser output coupler 18 such that air or material at that focus point 16 will breakdown into a micro-plasma or small spark. The preferred method is to use an eye-safe Class I laser operating in the 1.4 to 1.6 micrometer wavelength range. Such hand held lasers are know to those of ordinary skill in the art and will not be described further. This system allows the practitioners, clinical assistants and patients to forgo the use of protective eye-ware required for the use of non-eye-safe Class IV lasers. In this way the doctor or practitioner also may perform a traditional visual examination of the various colors and hues of suspected cancerous tissues without the masking and distortion often associated with wavelength band filter lenses typically employed in laser protective eye-ware.

A spectrophotometer 20 and plasma light collection optical fiber 22 are used to collect the light from each plasma spark and to generate the line spectra emission from the elements present at the targeted point on the tissue surface. The preferred method is to use a fiber spectrophotometer 20 with a plasma light collection optical fiber 22 close coupled to the laser focus point 16. An example includes integration of the plasma light collection optical fiber 22 into the laser focusing optical lens system shown at 14. A preferred design provides for the light collection optical fiber 22 to be integrated parallel to or at an angle to the optical axis of the laser pulse launching optics. The end of the collection optical fiber 22 may be polished flat and perpendicular or at an angle and with or without a radius curvature to facilitate plasma light collection. A small objective lens system, prism and/or protective optics preferably made of sapphire 24 may be added to the end of the collection optical fiber 22 to facilitate cleaning and fiber life.

A photodiode electronic signal is produced from the emission of the laser pulse. This laser pulse signal is used as T=0 to start a timing circuit and time delay for the spectrophotometer optical signal collection gate. The time delay and gate is optimized for collection of line spectra emission from the plasma during the period of the plasma's maximum emission intensity. The preferred design is a small hand held laser 10 with integrated delivery 14 and collection optics 22 and a portable pocket, briefcase or bench top sized laser power supply 26 and spectrophotometer 20 with computer interface and display 28. The computer interface and display 28 can be use to plot the results and provide instant analysis of the levels of selected elements. The emission intensity of a given element is directly related to its level in the target tissue at the laser focus point 16. The laser focus point 16 can be used to scan a tissue area of concern and to precisely outline the area containing cancerous cells.

Use of the system shown in FIG. 1 is illustrated in the laser plasma line spectra of normal and cancerous human skin, shown in FIGS. 2 and 3 respectively. FIG. 2 is a trace showing a line spectra emission of the elements calcium and aluminum from normal healthy skin surface cells using the present system. The peak 40, between 392 nanometers and 393 nanometers, represents the level of calcium while peak 42, between 396 and 397 nanometers, represents aluminum. The emission intensity is directly related to actual level of each element in the surface level cells. By way of contrast, FIG. 3 is a trace showing a line spectra emission of the elements calcium and aluminum from cancerous skin surface cells using the present system. It can be seen that in the cancerous tissue the cellular level of calcium, peak 44 is almost 2 fold greater than in FIG. 2, while the level of aluminum, peak 46, is largely unchanged. Using the present invention during a visual examination screening procedure for skin cancer the system is used to instantaneously confirm the presence or absence of cancerous cells without the need for a traditional biopsy. The system can also be used to map out the region of cancerous cells at the skin surface level. In use one can detect only one of the known marker elements, multiple marker elements, or marker elements plus internal control elements in determining the cancerous cells versus non-cancerous cells. The determination of cancerous versus non-cancerous can be made either from a determination of the increase or decrease of a maker element level relative to the expected level in a non-cancerous cell. This can be based on the levels found in adjacent non-cancerous cells or based on pre-determined levels based on experience from scanning non-cancerous cells. These expected levels may vary from species to species and between sexes. Alternatively, the internal control elements can be used as ratio factors allowing the comparison of the ratio of marker element value to the internal control element value in a test scan versus the same ratio expected in non-cancerous cells. Use of the procedure will permit refinement of the range of values of marker elements that are expected in non-cancerous cells thereby permitting increased accuracy of determination and possibly earlier detection of cancerous cells. The present invention is expected to find use with any species of plant or animal as a screening and surgical tool. Typically, it will be used in animal subjects, but may also find use with plant subjects. Plant subject use might occur to permit rapid screening of plant cells to determine if there has been transformation of the cells to cancerous cells. In the present specification and claims the term subject embraces both plant and animal subjects unless noted otherwise. The present invention finds special use in-situ in subjects, but could also be used on samples take from a subject.

During an excisional biopsy, the doctor or clinician will cut into deeper layers of the skin tissue, beyond the 50 micrometer depth, to remove the cancerous cells. The deeper layers of cells contain marker elements such as copper and sodium that exhibit large changes in their cellular levels when cells become cancerous. For example, in the deeper skin cell layers, copper cellular levels decrease by 10× while iron cellular levels remain relatively constant when the cell is cancerous. Thus, at deeper cell levels the element iron changes from a marker element in surface cells, ie. less than 50 micron depth, into an internal control element at the deeper cell levels. Also in these deeper skin layers sodium cellular levels increase by 3× while potassium cellular levels remain relatively constant in cancerous cells. Thus, cooper and sodium are marker elements for cancerous cells at deeper cell levels of more than 50 microns from the surface and iron and potassium are internal control elements in these deeper levels. During an excisional biopsy the margins of the excision are tested in-situ for the presence of skin cancer using the laser plasma spectroscopy system of the present invention and measuring one or more of the marker elements and may include measurement of the internal control elements if desired. For each targeted area, this provides the doctor or clinician with an instantaneous confirmation of the presence or absence of cancer cells at the margins without the need for a traditional biopsy staining and microscopic reading. Thus the present system will also find use during the excisional biopsy process to ensure that all and only the cancerous cells are removed and do so in real time.

The skin is the body's largest organ. The lungs breathe, and so does the skin. The breathing skin provides an exit for toxins and chemicals. As such this method and device may also be used for screening and detection of many other diseases, cancers, and abnormalities. Changes in the cellular levels of various other marker elements in the cells of the skin may be associated with the ingestion of drugs or poisons including by way of example only amphetamines and pesticides. Skin marker element composition changes may also result from the interaction of two or more prescription drugs. Changes in the marker element composition of the skin may be associated with numerous ailments including by way of example only abdominal cancer, anemia, back tumors, gastrointestinal bleeding, heart attack, hip cancer, anemia, leukemia, non-Hodgkin's lymphoma, diabetes and sickle cell anemia. It is believed that the present system can be used in conjunction with specific spectrophotometer wavelengths and software that are optimized to detect the specific changes in the levels of marker elements in the skin surface that are associated with a specific abnormality to lead to early detection of a variety of physiological changes or assaults to the body.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

We claim:

1. A method for detection of cancerous cells in a subject comprising the steps of:
a) directing a pulsed laser beam with greater than 0.1 megawatt per pulse output power at a plurality of cells of said subject to produce a plasma spark at the surface of said plurality of cells;
b) detecting a line spectra emission of at least one marker element resulting from the plasma spark;
c) determining a level of said at least one marker element based on the intensity of said emission; and
d) comparing the determined level to an expected level of said at least one marker element, wherein said expected level is the level found in a non-cancerous cell, and determining if there is a difference between said expected level of said at least one marker element and the determined level of said at least one marker element, wherein a difference between said expected level and the determined level is indicative of cancerous cells.

2. The method as recited in claim 1 wherein step a) comprises directing said laser beam at a plurality of skin surface cells.

3. The method as recited in claim 1 wherein step a) comprises directing said laser beam at a plurality of cells located at least 50 microns under a skin surface.

4. The method as recited in claim 1 wherein step b) comprises detecting a line spectra emission of at least one marker element selected from the group consisting of calcium, iron, zinc, copper, and sodium.

5. The method as recited in claim 1 wherein step a) comprises directing a class I eye-safe pulsed laser beam with greater than 0.1 megawatt per pulse output power at said plurality of cells.

6. The method as recited in claim 1 wherein step a) comprises use of a Q-switched pulsed laser.

7. The method as recited in claim 1 wherein step b) comprises detecting a line spectra emission of at least one marker element selected from the group consisting of calcium, iron, zinc, and sodium; and
wherein step d) comprises determining if the determined level is greater than the expected level.

8. The method as recited in claim 1 wherein step b) comprises detecting a line spectra emission of copper; and
wherein step d) comprises determining if the determined level of cooper is less than the expected level.

9. The method as recited in claim 1 comprising the further steps of directing said pulsed laser beam at a plurality of known non-cancerous cells of said subject to produce a plasma spark at the surface of said plurality of cells;
detecting a line spectra emission of at least one marker element resulting from the plasma spark;
determining a level of said at least one marker element based on the intensity of said emission; and
using the determined level as said expected level in step d).

10. The method as recited in claim 1 comprising the further steps after step d) of scanning a plurality of adjacent cells and mapping on said subject an area of cancerous cells.

11. The method as recited in claim 1 wherein said method is performed during an excisional biopsy of said subject at least once on cells at a margin of said biopsy to determine if cancerous cells are present in said margin.

12. The method as recited in claim 1 comprising the further steps of detecting a line spectra emission of at least one internal control element from said plurality of cells; determining the level of said at least one internal control element based on the intensity of said emission; calculating a ratio of the determined level of said at least one marker element to the determined level of said at least one internal control element in said plurality of cells; determining if there is a difference between said ratio and an expected ratio of said at least one marker element to said at least one internal control element in non-cancerous cells, wherein a difference between said ratios is indicative of cancerous cells.

13. The method as recited in claim 12 wherein said at least one internal control element is selected from the group consisting of aluminum, potassium, magnesium, and iron.

14. The method as recited in claim 12 comprising the further steps of detecting a line spectra emission of at least one internal control element and at least one marker element from a plurality of known non-cancerous cells of said subject; determining the levels of said at least one internal control element and said at least one marker element based on the intensity of said emissions; calculating a ratio of the determined level of said at least one marker element to the determined level of said at least one internal control element in said plurality of known non-cancerous cells; and using said determined ratio from said known non-cancerous cells as said expected ratio.

15. The method as recited in claim 1 wherein step a) comprises directing said laser beam at a plurality of cells in-situ of said subject.
